(19) United States
(12) Patent Application Publication
(10) Pub. No.: US 2022/0401703 A1
(43) Pub. Date: Dec. 22, 2022

(54) Vascular Access Systems and Methods Thereof

(71) Applicant: Bard Peripheral Vascular, Inc., Franklin Lakes, NJ (US)

(72) Inventors: Nathan GOOCH, Centerville, UT (US); Bradley FORSYTH, Sandy, UT (US)

(21) Appl. No.: 17/763,154

(22) PCT Filed: Sep. 23, 2019
(86) PCT No.: PCT/US19/52477

§ 371 Date: Mar. 23, 2022

(51) Int. Cl. A61M 25/06; A61M 39/02; A61M 25/00; A61M 25/01

(52) U.S. Cl. CPC A61M 25/065; A61M 39/0208; A61M 25/0097; A61M 25/0108

(57) ABSTRACT

Disclosed herein are vascular access systems and methods thereof. For example, a vascular access system includes a port configured for subdermal implantation and a single-use access device. The port can include a port hub and a catheter tube. The port hub can include an access funnel and a septum defining a pair of port-hub lumens. The catheter tube can include a septum defining a pair of catheter-tube lumens fluidly coupled to the pair of port-hub lumens. The access device can include a bifurcated hub and a pair of cannulas. The pair of cannulas defines a pair of cannula lumens coupled to a pair of access device-hub lumens of the bifurcated hub. The pair of cannulas can be configured to simultaneously insert into the access funnel and couple the pair of cannula lumens to the pair of catheter-tube lumens when the pair of cannulas is fully seated in the port hub.

### BACKGROUND

A non-cuffed hemodialysis catheter is often used in an emergency or for a relatively short period of time (e.g., <3 weeks) when a patient is in immediate need of hemodialysis due to kidney failure. Likewise, a cuffed hemodialysis catheter is often used when a patient is in immediate need of hemodialysis, but the cuffed hemodialysis catheter can be used for a relatively long period of time (e.g., >3 weeks). Both types of hemodialysis catheter are typically used until permanent vascular access can be established by way of an arteriovenous (“AV”) fistula or graft, each of which require surgery and recovery time. Once placed, such hemodialysis catheters have an extracorporeal portion that requires routine maintenance to avoid clots and infection. In addition, there is a risk of the patient or others tampering with the extracorporeal portion of such hemodialysis catheters, which can lead to air embolism. In view of the foregoing, there is a need for vascular access systems and methods thereof that obviate the routine maintenance and the risk of tampering inherent in existing hemodialysis catheters.

Disclosed herein are vascular access systems and methods thereof that address at least the foregoing need.

### SUMMARY

Disclosed herein is vascular access system including, in some embodiments, a port and a single-use access device. The port is configured for subdermal implantation. The port includes a port hub and a catheter tube. The port hub has an access funnel and a septum defining a pair of port-hub lumens. The catheter tube has a septum defining a pair of catheter-tube lumens fluidly coupled to the pair of port-hub lumens. The access device includes a bifurcated hub and a pair of cannulas. The bifurcated hub includes a pair of access device-hub lumens. The pair of cannulas defines a pair of cannula lumens fluidly coupled to the pair of access device-hub lumens. The pair of cannulas is configured to simultaneously insert into the access funnel and fluidly couple the pair of cannula lumens to the pair of catheter-tube lumens when the pair of cannulas is fully seated in the port hub.

In some embodiments, the pair of cannulas is configured to pierce skin of a patient before inserting the pair of cannulas into the access funnel of the port when implanted under the skin.

In some embodiments, the single-use access device includes a pair of extension legs defining a pair of extension-leg lumens fluidly coupled to the pair of access device-hub lumens.

In some embodiments, the port hub has a pair of valves in a distal-end portion of the access funnel configured to open when the pair of cannulas is inserted into the pair of valves.

In some embodiments, the port hub has a bifurcated stem extending from the port hub configured to insert into the pair of catheter-tube lumens.

In some embodiments, the port includes a catheter lock over the catheter tube over the bifurcated stem. The catheter lock is configured to lock the catheter tube onto the bifurcated stem.

In some embodiments, the vascular access system is configured for continuous flow at a flow rate of at least 300 to 500 mL/min.

Also disclosed herein is a port configured for subdermal implantation including, in some embodiments, a port hub and a catheter tube. The port hub has an access funnel and a septum defining a pair of port-hub lumens. The catheter tube has a septum defining a pair of catheter-tube lumens fluidly coupled to the pair of port-hub lumens.

In some embodiments, the port hub has a pair of valves in a distal-end portion of the access funnel configured to open when a pair of cannulas of a single-use access device is inserted into the pair of valves.

In some embodiments, the port hub has a bifurcated stem extending from the port hub configured to insert into the pair of catheter-tube lumens.

In some embodiments, the port includes a catheter lock over the catheter tube over the bifurcated stem. The catheter lock is configured to lock the catheter tube onto the bifurcated stem.

In some embodiments, the catheter tube is a radiopaque polyurethane catheter tube.

In some embodiments, the access funnel is titanium or an alloy thereof. The access funnel is configured for a relatively low-angle approach by a single-use access device.

In some embodiments, the access funnel is disposed within a silicone body of the port hub.

Also disclosed herein is a single-use access device including, in some embodiments, a bifurcated hub and a pair of cannulas. The bifurcated hub includes a pair of access device-hub lumens. The pair of cannulas define a pair of cannula lumens fluidly coupled to the pair of access device-hub lumens. The pair of cannulas is configured to simultaneously insert into an access funnel of an implantable port and fluidly couple the pair of cannula lumens to a pair of catheter-tube lumens when the pair of cannulas is fully seated in the implantable port.

In some embodiments, the pair of cannulas is configured to pierce skin of a patient before inserting the pair of cannulas into the access funnel of the port when implanted under the skin.

In some embodiments, the access device further includes a pair of extension legs defining a pair of extension-leg lumens fluidly coupled to the pair of access device-hub lumens.

In some embodiments, the access device further includes a pair of Luer connectors. Each Luer connector is connected to an extension leg of the pair of extension legs.

Also disclosed herein is a method of vascular access system including, in some embodiments, a step of locating an access site and identifying an implanted port implanted in a patient at the access site by palpation of the patient. The port includes a port hub and a catheter tube. The port hub has an access funnel and a septum defining a pair of port-hub lumens. The catheter tube has a septum defining a pair of catheter-tube lumens fluidly coupled to the pair of port-hub lumens. The method also includes a step of cleaning and disinfecting the access site with an antiseptic for skin disinfection. The method also includes a step of preparing a single-use access device including priming the access device with saline. The access device includes a bifurcated hub and a pair of cannulas. The bifurcated hub includes a pair of access device-hub lumens. The pair of cannulas defines a pair of cannula lumens fluidly coupled to the pair of access device-hub lumens. The method also includes a step of stabilizing the implanted port with a non-dominant, sterile-gloved hand. The method also includes a step of simultaneously inserting the pair of cannulas into the access funnel until the pair of cannulas is fully seated in the port hub, thereby fluidly coupling the pair of cannula lumens to the pair of catheter-tube lumens.

In some embodiments, inserting the pair of cannulas into the access funnel includes piercing skin of the patient and inserting the pair of cannulas into the access funnel with a shallow angle of approach of less than about 30° between the patient and the pair of cannulas.

In some embodiments, the method further includes a step of aspirating the vascular access system for blood flow. The method also includes a step of securely dressing the access site with a wound dressing.

These and other features of the concepts provided herein will become more apparent to those of skill in the art in view of the accompanying drawings and following description, which describe particular embodiments of such concepts in greater detail.

### DRAWINGS

FIG. 1 illustrates a vascular access system including a port and an access device disposed in the port in accordance with some embodiments.

FIG. 2 illustrates the vascular access system of FIG. 1 with the access device separated from the port in accordance with some embodiments.

FIG. 3 illustrates a cross-section of a port hub of the port in accordance with some embodiments.

FIG. 4 illustrates an exploded view of the port hub in accordance with some embodiments.

FIG. 5 illustrates a close-up view of a distal-end portion of the port hub in accordance with some embodiments.

FIG. 6 illustrates a cross-section of a catheter tube of the port in accordance with some embodiments.

FIG. 7 illustrates a detailed view of a pair of cannulas of the access device in accordance with some embodiments.

FIG. 8 illustrates the port implanted in a patient in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need for vascular access systems and methods thereof that obviate the routine maintenance and the risk of tampering inherent in existing hemodialysis catheters. Disclosed herein are vascular access systems and methods thereof that address at least the foregoing need.

### Vascular Access Systems

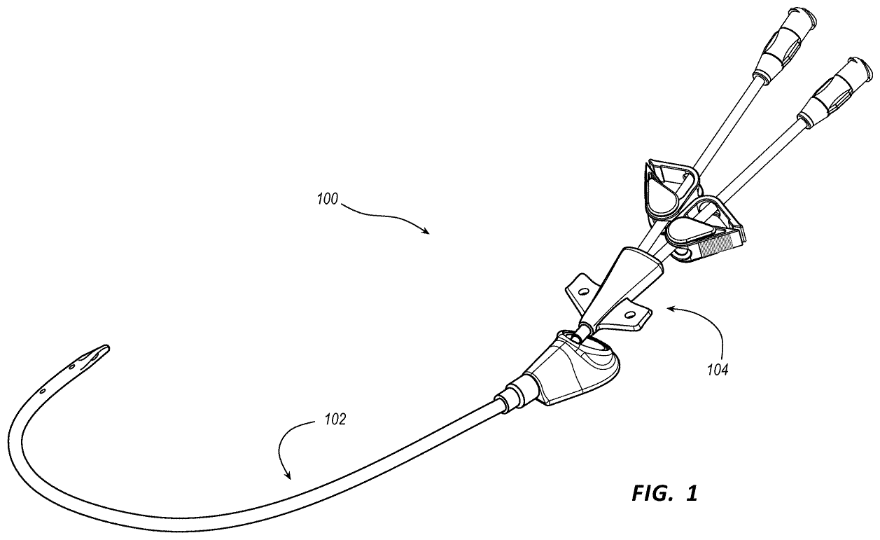

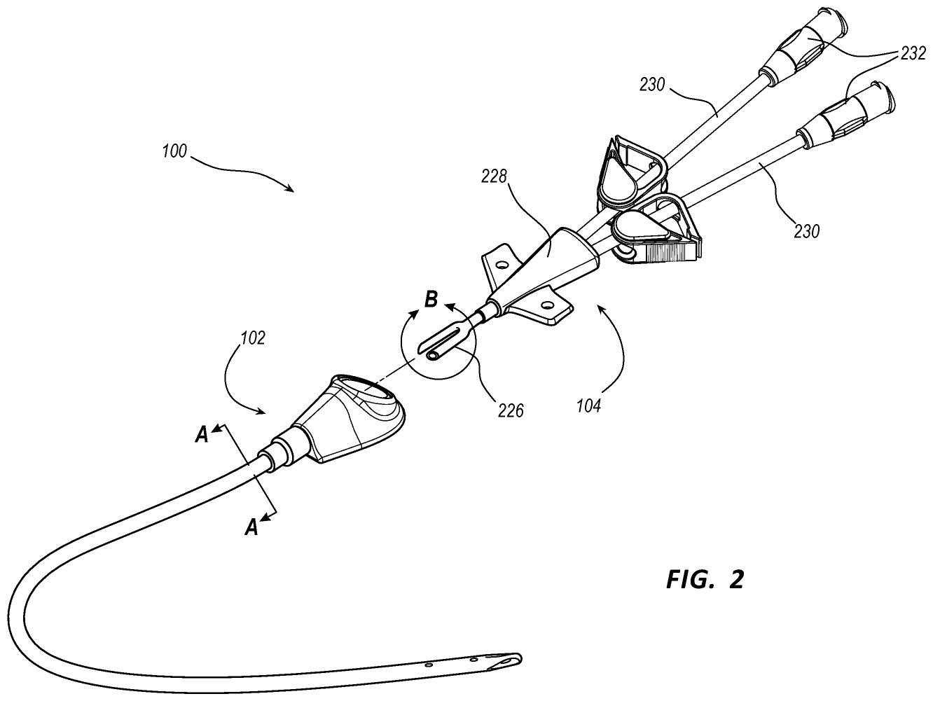

FIG. 1 illustrates a vascular access system 100 including a port 102 and an access device 104 disposed in the port 102 in accordance with some embodiments. FIG. 2 illustrates the vascular access system 100 with the access device 104 separated from the port 102 in accordance with some embodiments.

As shown, the vascular access system 100 includes port 102 and the access device 104. The vascular access system 100 is configured such that the access device 104 can be inserted into the port 102 and removed from the port a number of different times; however, the access device 104 is a single-use access device in that while the port 102 is implanted in a patient, an unused access device 104 should be used each time the access device 104 is inserted into the port 104.

The vascular access system 100 is bi-luminal in that each component of the port 102 and the access device 104 includes two fluid passageways therethrough. Notably, the vascular access system 100 is configured such that the two fluid passageways of the port 102 and the two fluid passageways of the access device 104 fluidly connect upon a single insertion of the access device 104 into the port 102 to form two fluid passageways through the vascular access system 100. A first fluid passageway of the two fluid passageways is configured as an arterial lumen to convey blood away from a heart of a patient. A second fluid passageway of the two fluid passageways is configured as a venous lumen to convey blood to the heart of the patient.

The vascular access system 100 is configured for continuous flow at a flow rate of at least about 300 to 500 mL/min, which is required for hemodialysis.

Additional description for the vascular access system 100 is set forth below with respect to the port 102 and the access device 104.

### Ports

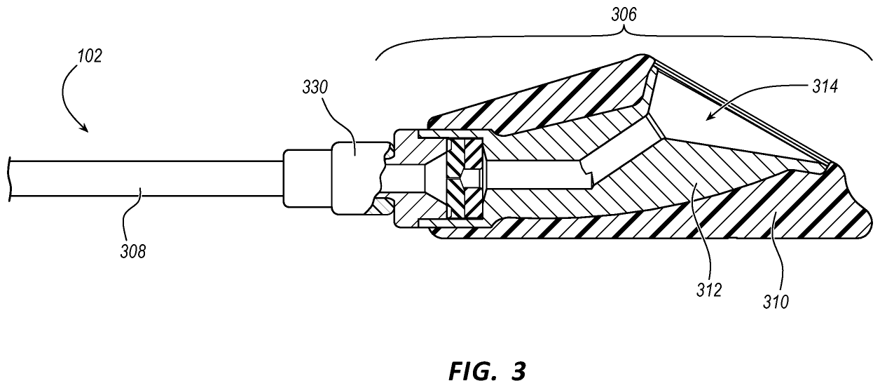

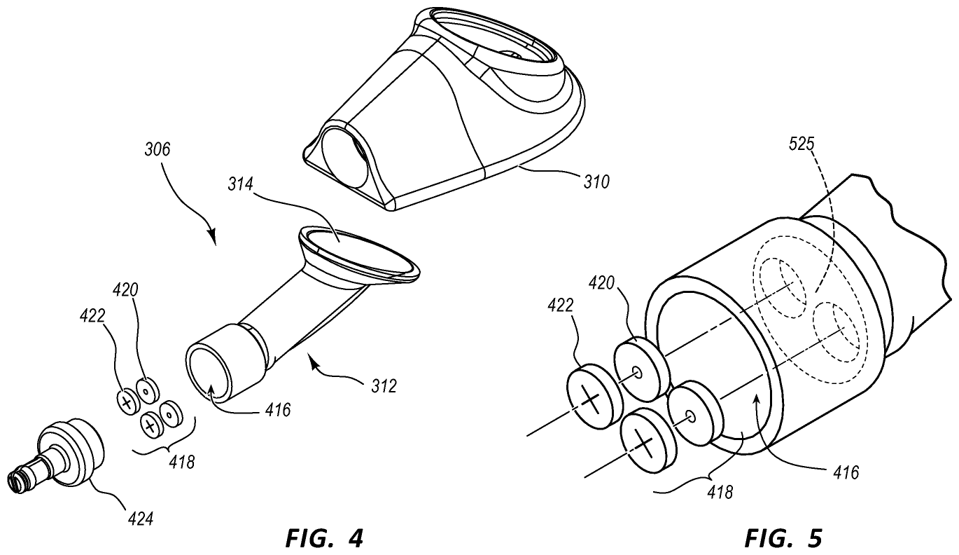

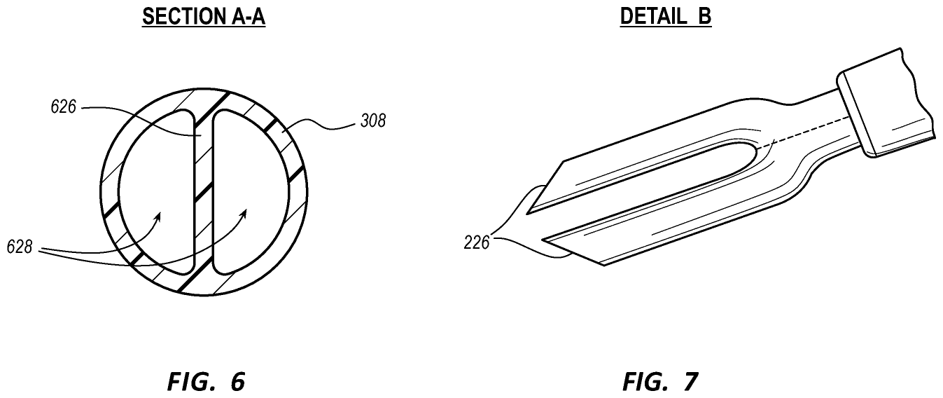

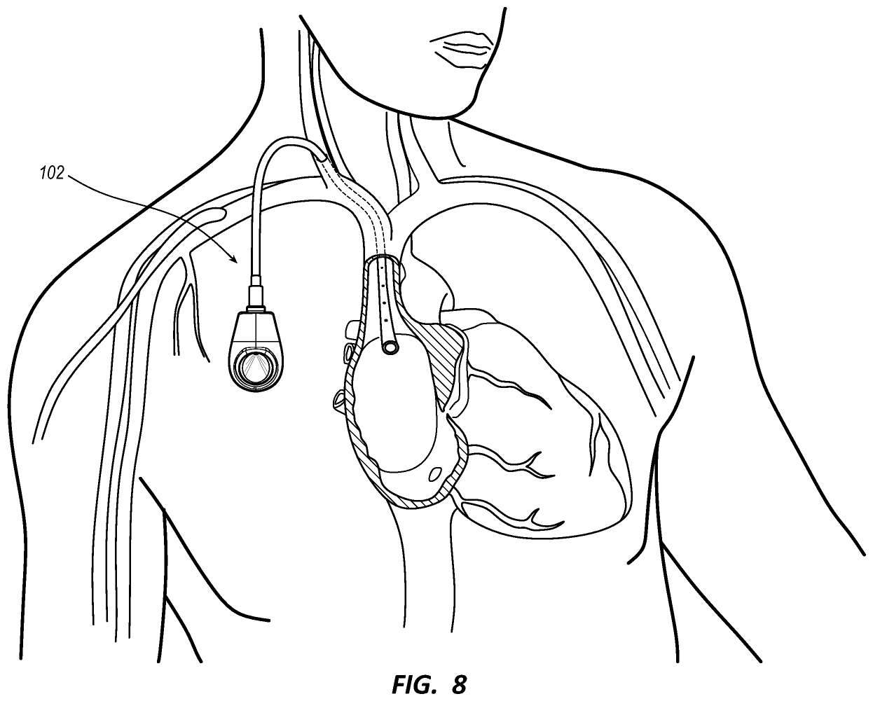

FIG. 3 illustrates a cross-section of a port hub 306 of the port 102 in accordance with some embodiments. FIG. 4 illustrates an exploded view of the port hub 306 in accordance with some embodiments. FIG. 5 illustrates a close-up view of a distal-end portion of the port hub in accordance with some embodiments. FIG. 6 illustrates a cross-section of a catheter tube 308 of the port in accordance with some embodiments. FIG. 8 illustrates the port 102 implanted in a patient in accordance with some embodiments.

As shown, the port 102 includes the port hub 306 and the catheter tube 308.

The port hub 306 includes a body 310 of a medically acceptable polymer having a relatively low durometer such as silicone. The body 310 of such a polymer provides patient comfort when the port 102 is implanted under the subdermis of a patient.

The port hub 306 includes an access funnel 312 disposed in the body 310 configured for a shallow angle of approach or insertion of the access device 104 of less than about 30° between the patient and the pair of cannulas 226 of the access device 104. (See FIG. 7 for the pair of cannulas 226 of the access device 104.) A relatively wide opening 314 in a proximal-end portion of the access funnel 312 is configured to catch the pair of cannulas 226 and direct the pair of cannulas 226 into a relatively narrow passageway of the access funnel 312 toward a distal-end portion 416 of the access funnel 312. The access funnel 312 can be titanium or an alloy thereof, which provides the access funnel 312 durability for the number of different times the access device 104 is anticipated to be inserted into the port 102.

The port hub 306 includes a pair of valves 418 disposed in the distal-end portion of the access funnel 312 configured to open when the pair of cannulas 226 of the single-use access device 104 is inserted into the pair of valves 481. Each valve of the pair of valves 418 can include two septums (e.g., silicone septums) sandwiched together such as holed septum 420 and slitted septum 422.

The port hub 306 includes a bifurcated stem 424 extending from the port hub 306 configured to insert into the pair of catheter-tube lumens 628. (See FIG. 6 for the pair of catheter-tube lumens 628 of the catheter tube 308.) While not shown, a septum proximally extending from the bifurcated stem 424 in accordance with the bifurcation of the bifurcated stem 424 abuts a septum 525 of the port hub 306 defining a pair of port-hub lumens configured to direct fluid flow from the pair of cannula lumens (see description set forth below) of the pair of cannulas 226 into the pair of catheter-tube lumens 628.

The catheter tube 308 includes a septum 626 defining a pair of catheter-tube 628 lumens fluidly coupled to the pair of port-hub lumens when the catheter tube 308 is disposed over the bifurcated stem 424. The catheter tube 308 can have a suitable length for tunneling the catheter tube 308 from a subclavicular access site to a right atrium of a heart. The catheter tube 308 can be a radiopaque polyurethane catheter tube of less than about 10 Fr such as 9 Fr or a catheter size in between (e.g., 9.6 Fr).

The port 102 further includes a catheter lock 330 configured to lock the catheter tube 308 onto the bifurcated stem 424.

### Access Devices

FIG. 2 illustrates the vascular access system 100 with the access device 104 separated from the port 102 in accordance with some embodiments. FIG. 7 illustrates a detailed view of a pair of cannulas 226 of the access device 104 in accordance with some embodiments.

As shown, the access device 104 includes the pair of cannulas 226, a bifurcated hub 228, a pair of extension legs 230, and a pair of Luer connectors 232 operably connected in the foregoing order.

The pair of cannulas 226 define a pair of cannula lumens. As set forth above, the pair of cannulas 226 is configured to simultaneously insert into the access funnel 312 of the port hub 306 and fluidly couple the pair of cannula lumens to the pair of catheter-tube lumens when the pair of cannulas 226 is fully seated in the port hub 306. When the port 102 is implanted under the subdermis of a patient, the pair of cannulas 226 is also configured to pierce skin of the patient before insertion into the access funnel 312. Accordingly, each cannula of the pair of cannulas 226 is configured with a beveled tip to pierce the skin of the patient. The pair of cannulas 226 can be a metal or an alloy such as stainless steel, which provides the pair of cannulas 226 sufficient rigidity to pierce the skin of the patient and insert into the access funnel 312.

The bifurcated hub 228 includes a pair of access device-hub lumens fluidly coupled to the pair of cannula lumens of the pair of cannulas 226.

The pair of extension legs 230 define a pair of extension-leg lumens fluidly coupled to the pair of access device-hub lumens of the bifurcated hub 228.

The pair of Luer connectors 232 define a pair of Luer-connector lumens fluidly coupled to the pair of extension-leg lumens of the pair of extension legs 230.

### Methods

A method of vascular access system includes a step of locating an access site and identifying an implanted port such as the port 102 implanted in a patient at the access site by palpation of the patient. The method also includes a step of cleaning and disinfecting the access site with an antiseptic (e.g., iodopovidone) for skin disinfection. The method also includes a step of preparing the access device 104 including priming the access device 104 with saline. The method also includes a step of stabilizing the implanted port 102 with a non-dominant, sterile-gloved hand. The method also includes a step of simultaneously inserting the pair of cannulas 226 into the access funnel 312 until the pair of cannulas 226 is fully seated in the port hub 306, thereby fluidly coupling the pair of cannula lumens to the pair of catheter-tube lumens 628. Inserting the pair of cannulas 226 into the access funnel 312 includes piercing skin of the patient and inserting the pair of cannulas 226 into the access funnel 312 with a shallow angle of approach of less than about 30° between the patient and the pair of cannulas 226. The method also includes a step of aspirating the vascular access system 100 for blood flow, as well as a step of securely dressing the access site with a wound dressing.

The vascular access system 100 leverages benefits of a fully subcutaneous port, which allows patients the ability to bathe or shower without inconveniencing the patients or adversely affecting their ports. Indeed, no portion of the port 102 is subject to exposure except during a hemodialysis procedure when the access device 104 is being used. Being a fully subcutaneous port improves cosmesis and obviates routine maintenance otherwise required to avoid clots and infection.

The vascular access system 100 also leverages benefits of the access device 104 looking and operating much like a standard hemodialysis catheter once the access device 104 is seated in the port 102. For example, the pair of extension legs 230 should appear familiar to clinicians and take little to no training to connect to other hemodialysis equipment.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A vascular access system, comprising:
a port configured for subdermal implantation, the port including:
a port hub having an access funnel and a septum defining a pair of port-hub lumens; and
a catheter tube having a septum defining a pair of catheter-tube lumens fluidly coupled to the pair of port-hub lumens; and
a single-use access device, the access device including:
a bifurcated hub including a pair of access device-hub lumens; and
a pair of cannulas defining a pair of cannula lumens fluidly coupled to the pair of access device-hub lumens, the pair of cannulas configured to simultaneously insert into the access funnel and fluidly couple the pair of cannula lumens to the pair of catheter-tube lumens when the pair of cannulas is fully seated in the port hub.

2. The vascular access system of claim 1, the pair of cannulas configured to pierce skin of a patient before inserting the pair of cannulas into the access funnel of the port when implanted under the skin.

3. The vascular access system of claim 1, the single-use access device including a pair of extension legs defining a pair of extension-leg lumens fluidly coupled to the pair of access device-hub lumens.

4. The vascular access system of claim 1, the port hub having a pair of valves in a distal-end portion of the access funnel configured to open when the pair of cannulas is inserted into the pair of valves.

5. The vascular access system of claim 1, the port hub having a bifurcated stem extending from the port hub configured to insert into the pair of catheter-tube lumens.

6. The vascular access system of claim 5, the port including a catheter lock over the catheter tube over the bifurcated stem, the catheter lock configured to lock the catheter tube onto the bifurcated stem.

7. The vascular access system of claim 1, the vascular access system configured for continuous flow at a flow rate of at least 300 to 500 mL/min.

8. A port configured for subdermal implantation, comprising:
a port hub having an access funnel and a septum defining a pair of port-hub lumens; and
a catheter tube having a septum defining a pair of catheter-tube lumens fluidly coupled to the pair of port-hub lumens.

9. The port of claim 8, the port hub having a pair of valves in a distal-end portion of the access funnel configured to open when a pair of cannulas of a single-use access device is inserted into the pair of valves.

10. The port of claim 8, the port hub having a bifurcated stem extending from the port hub configured to insert into the pair of catheter-tube lumens.

11. The port of claim 10, the port including a catheter lock over the catheter tube over the bifurcated stem, the catheter lock configured to lock the catheter tube onto the bifurcated stem.

12. The port of claim 8, wherein the catheter tube is a radiopaque polyurethane catheter tube.

13. The port of claim 8, wherein the access funnel is titanium or an alloy thereof, the access funnel configured for a relatively low-angle approach by a single-use access device.

14. The port of claim 8, wherein the access funnel is disposed within a silicone body of the port hub.

15. A single-use access device, comprising:
a bifurcated hub including a pair of access device-hub lumens; and
a pair of cannulas defining a pair of cannula lumens fluidly coupled to the pair of access device-hub lumens, the pair of cannulas configured to simultaneously insert into an access funnel of an implantable port and fluidly couple the pair of cannula lumens to a pair of catheter-tube lumens when the pair of cannulas is fully seated in the implantable port.

16. The access device of claim 15, the pair of cannulas configured to pierce skin of a patient before inserting the pair of cannulas into the access funnel of the port when implanted under the skin.

17. The access device of claim 15, further comprising a pair of extension legs defining a pair of extension-leg lumens fluidly coupled to the pair of access device-hub lumens.

18. The access device of claim 17, further comprising a pair of Luer connectors, each Luer connector connected to an extension leg of the pair of extension legs.

19-21. (canceled)
